(19) United States
(12) Patent Application Publication
(10) Pub. No.: US 2019/0083727 A1
(43) Pub. Date: Mar. 21, 2019

(54) Removable Fiberoptic Adapter for Difficult Intubation with Laryngeal Mask Airway

(71) Applicants: Elena Anigati, Chalfont, PA (US); Carol Anigati, Gwynedd, PA (US)

(72) Inventors: Elena Anigati, Chalfont, PA (US); Carol Anigati, Gwynedd, PA (US)

(21) Appl. No.: 16/043,405

(22) Filed: Jul. 24, 2018

Related U.S. Application Data
(60) Provisional application No. 62/559,973, filed on Sep. 18, 2017.

(51) Int. Cl. A61M 16/04

(52) U.S. Cl. CPC A61M 16/045; A61M 16/047; A61B 1/00105; A61M 16/0463; A61M 16/0488

(57) ABSTRACT

A removable fiberoptic adapter is secured onto a respiratory-device, such as a laryngeal mask airway (LMA) or a ventilating airway mask, to allow for endoscopes to be inserted through the LMA while maintaining a connection to an oxygen source to sustain the patient as the patient breathes. The removable fiberoptic adapter includes an airway tube, an oxygen port, a carbon dioxide outlet, an implement port, and an adapter plug. The oxygen port and the carbon dioxide outlet allow for the introduction of air or oxygen and the removal of carbon dioxide for respiration of the patient. The oxygen port and the carbon dioxide outlet are laterally connected to the airway tube. The implement port allows for the insertion of endoscopes or other medical implements through the respiratory-device. The adapter plug engages the implement inlet in order to restrict fluid flow through the implement inlet.

The current application claims a priority to the U.S. Provisional Patent application Ser. No. 62/559,973 filed on Sep. 18, 2017.

### FIELD OF THE INVENTION

The present invention relates generally to an adapter for a laryngeal mask airway (LMA) tube used for intubating a patient or for a ventilating face mask used for non-invasive ventilation. More specifically, the present invention utilizes an adapter that is capable of selectively engaging with the external end of the LMA tube or face mask cuff.

### BACKGROUND OF THE INVENTION

Typical laryngeal mask airway (LMA) tubes are utilized for intubating a patient to maintain an open airway while the patient is under anesthesia or unconscious. The LMA tube forms an airtight seal against the glottis through the inclusion of an inflatable or self-sealing cuff. A common use for LMA tubes is to allow oxygen or anesthesia to be channeled into a patient's lungs for unconscious patients during surgery or emergency medical response situations. The LMA typically is additionally able to be implemented during bronchoscopies and a plurality of airway procedures; however, the channeling of oxygen or anesthesia is not simultaneously implemented during the bronchoscopy or other airway procedures.

Ventilating face masks are utilized for non-invasive ventilation applications. The ventilating face mask is tightly fitted on the face of a patient. The ventilating face mask forms a seal with the patient's face without the need for tracheal intubation. The ventilating face mask typically incorporates an adapter cuff to attach the ventilating face mask with an air and/or anesthesia supply to introduce the respective gas to the patient.

The present invention is an adapter suited to detachably engage with a respiratory-device, such as the LMA tube or the adapter cuff of a ventilating face mask. The present invention streamlines the process of inserting fiber-optic scopes and endotracheal tubes therein. Once the fiber-optic scope and the endotracheal tube are inserted, the adapter may be opened and disengaged from the LMA tube or the adapter cuff, allowing the users greater flexibility in positioning said tubes within the LMA tube or the adapter cuff.

### BRIEF DESCRIPTION OF THE DRAWINGS

FIG. 1 is a perspective view of the present invention.

FIG. 2 is a front view of the present invention, wherein the adapter plug is disengaged from the implement port.

FIG. 3 is a rear view of the present invention, wherein the adapter plug is disengaged from the implement port.

FIG. 4 is a top view of the present invention, wherein the adapter plug is removed.

FIG. 5 is a bottom view of the present invention.

FIG. 6 is a right view of the present invention.

FIG. 7 is a left view of the present invention.

FIG. 8 is a cross-sectional view of the present invention along the line 8-8 shown in FIG. 2.

### DETAIL DESCRIPTIONS OF THE INVENTION

All illustrations of the drawings are for the purpose of describing selected versions of the present invention and are not intended to limit the scope of the present invention.

The present invention is a removable fiberoptic adapter for a respiratory-device, such as a laryngeal mask airway (LMA) or a ventilating face mask. The present invention is affixed to the respiratory-device for implementation. The LMA maintains an open airway for a plurality of airway procedures: to provide oxygen, to administer anesthesia, or to receive an endoscope. The LMA is positioned through the patient's trachea and adjacent to the patient's glottis to form a hermetic seal around the perimeter of the trachea to provide a channel for air and anesthesia to be introduced to sustain or sedate the patient. The ventilating face mask creates a seal around the patient's mouth and nose, such that tracheal intubation is not needed to channel air and/or anesthesia to sustain or sedate the patient, respectively. The present invention allows for medical devices, such as an endoscope to be inserted, interchanged, or removed quickly through an exposed end of the LMA or an adapter cuff of the ventilating face mask.

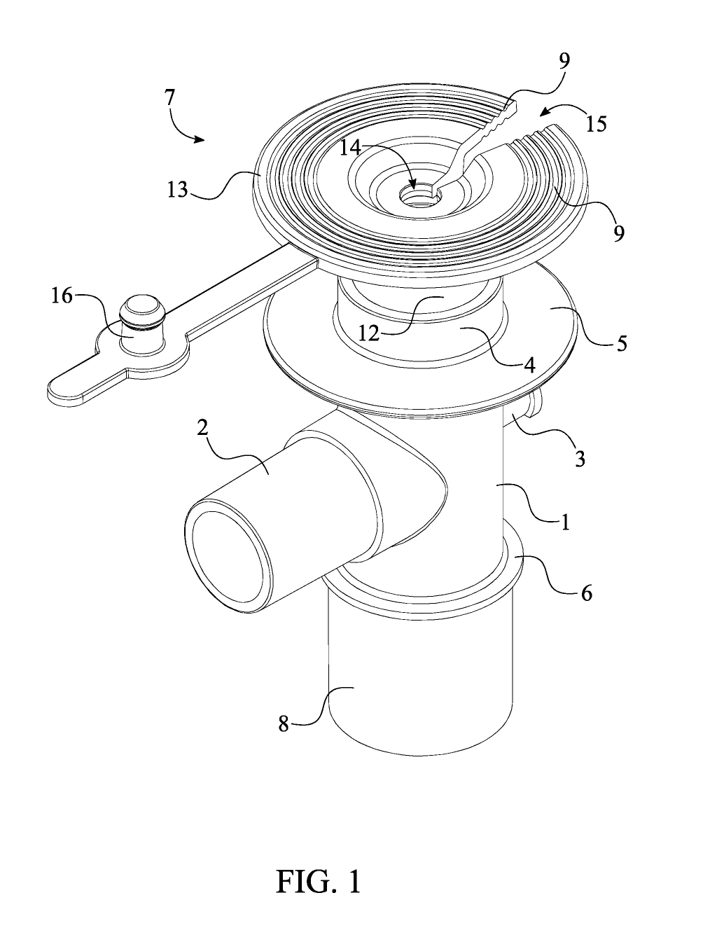

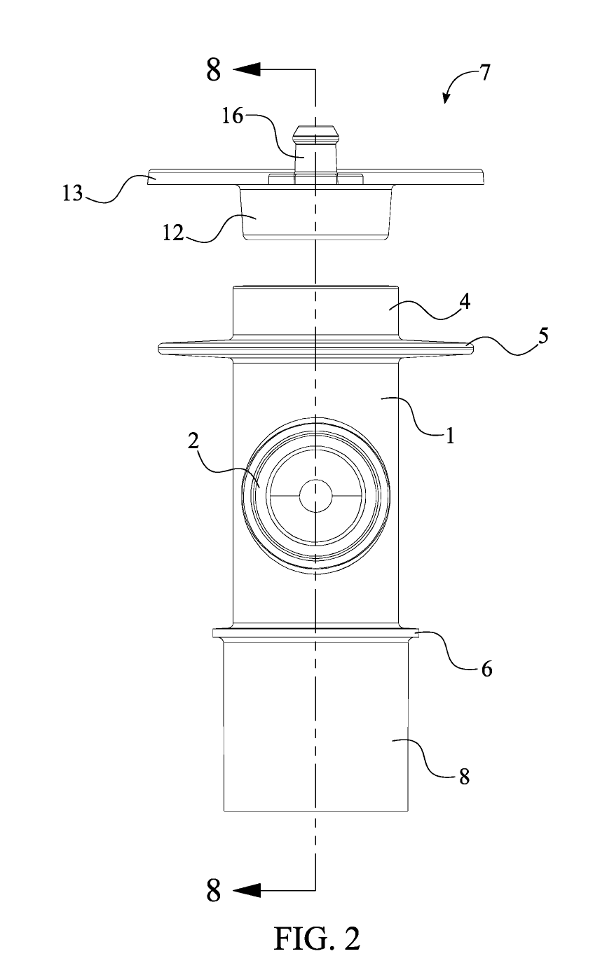

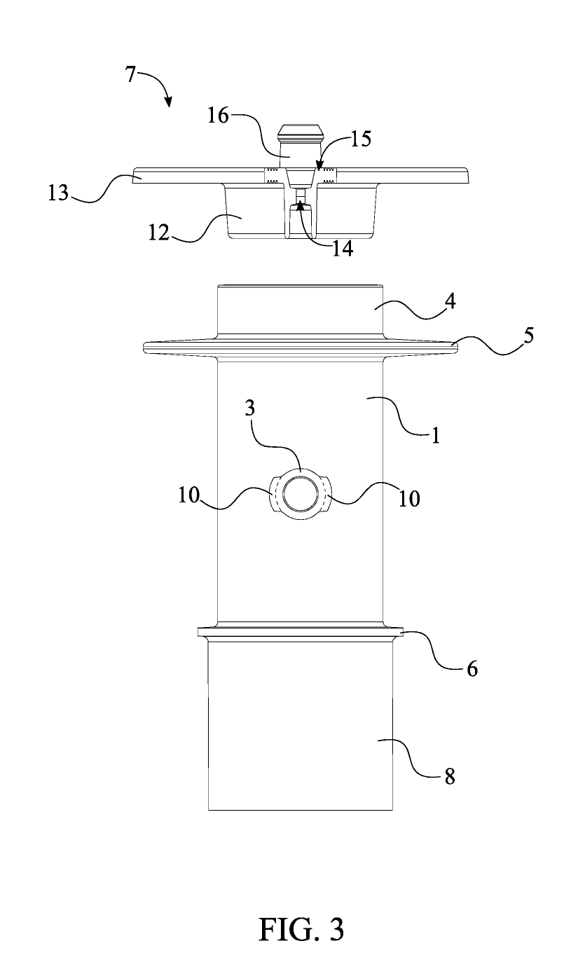

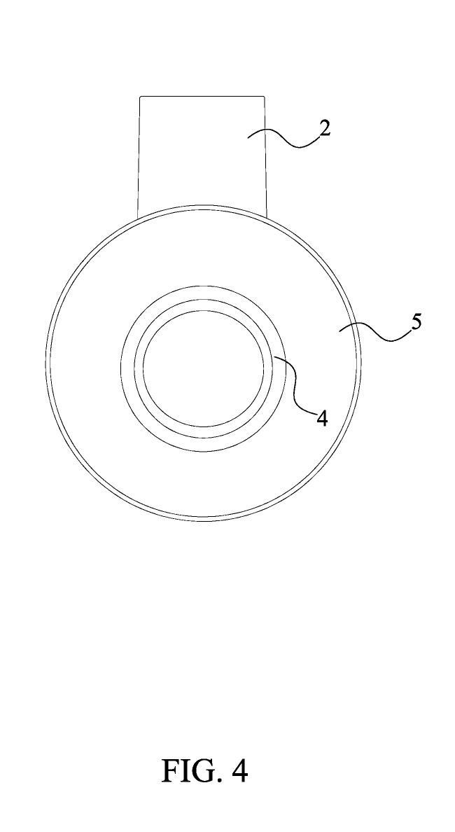

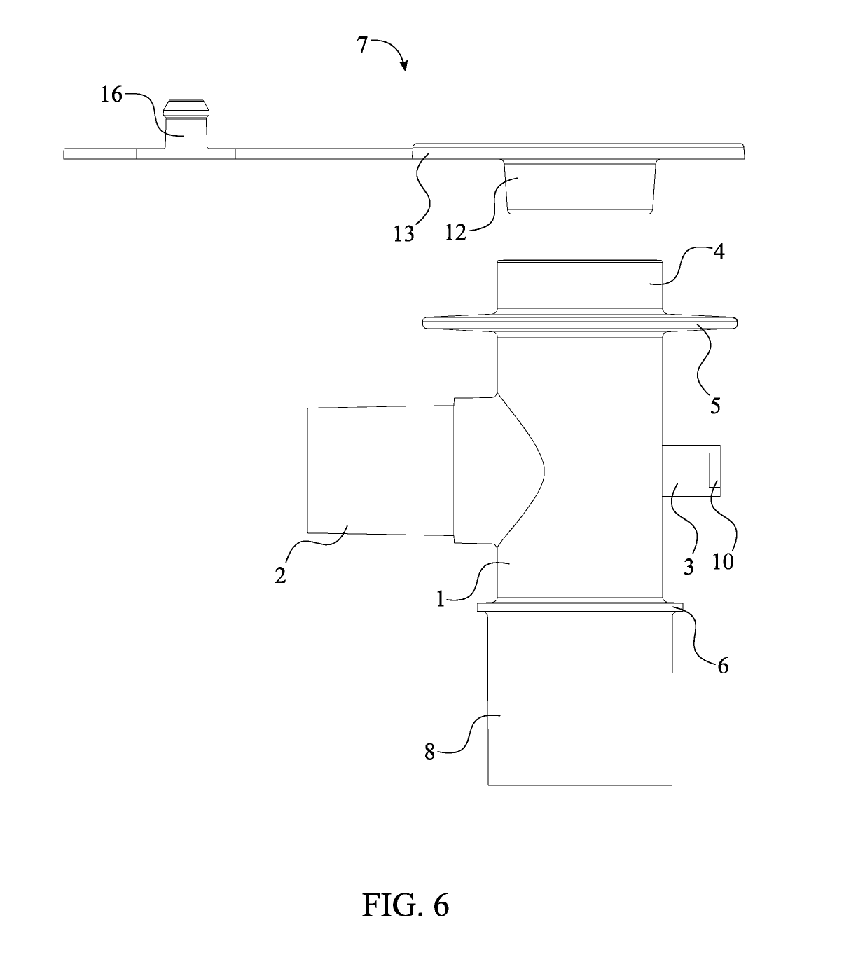

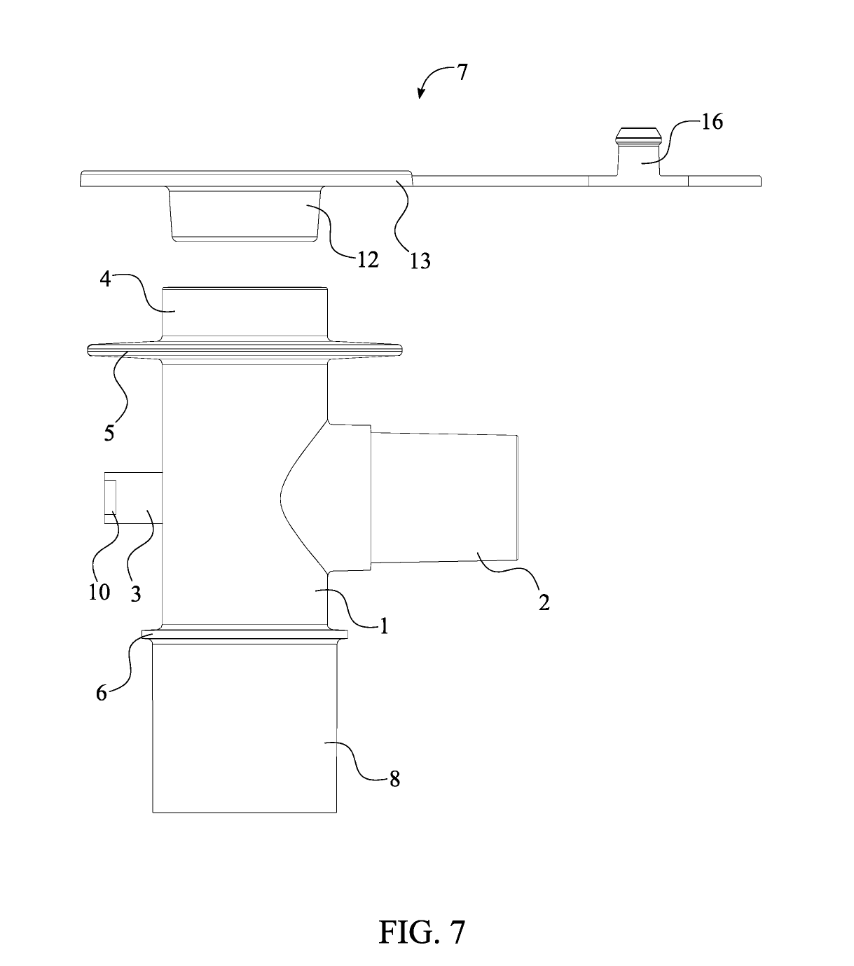

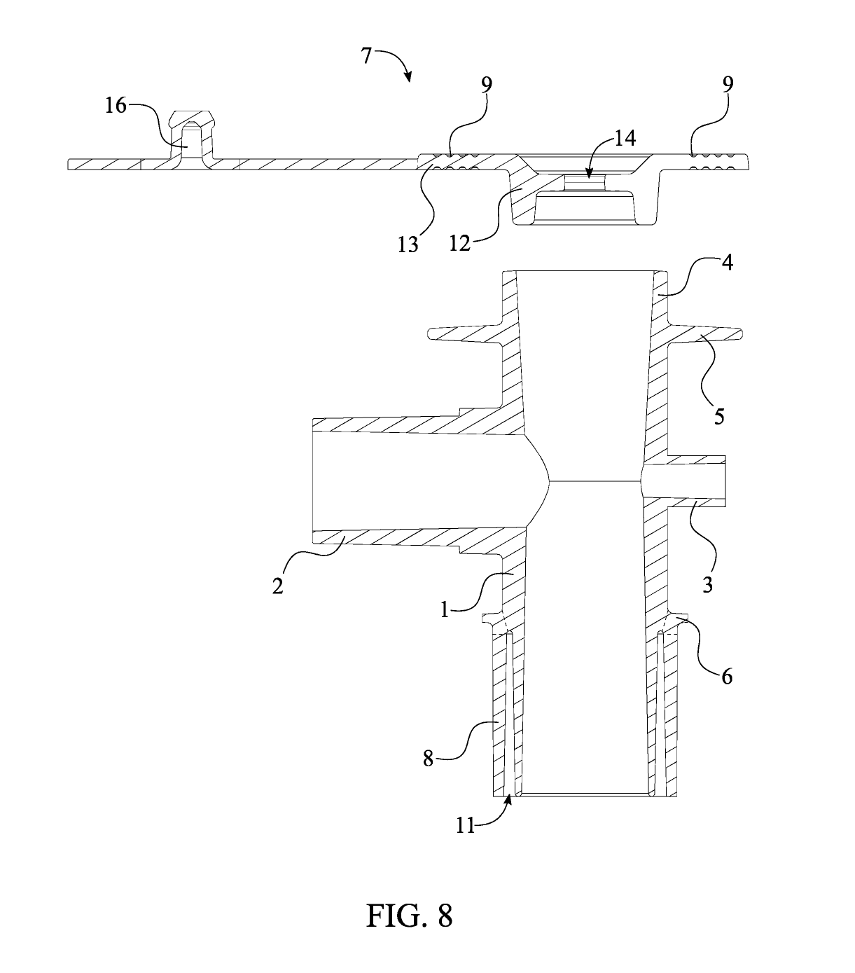

In accordance to FIG. 1, the present invention comprises an airway tube 1, an oxygen port 2, a carbon dioxide outlet 3, a grasping flange 5, an implement port 4, a mounting flange 6, an adapter plug 7, and an adapter mounting tube 8. The airway tube 1 is a channel that allows for oxygen and carbon dioxide to flow into and out from the patient as well as for the insertion of medical devices, such as an endoscope. The oxygen port 2 is coupled with an external oxygen source to allow oxygen to be introduced to the patient's body and sustain the patient. The oxygen port 2 is laterally connected to the airway tube 1, shown in FIG. 6 and FIG. 7. The oxygen port 2 is in fluid communication with the airway tube 1 to allow oxygen to flow through the oxygen port 2 into the airway tube 1, detailed in FIG. 8. The carbon dioxide outlet 3 allows for the removal of carbon dioxide generated from the patient's respiration. The carbon dioxide outlet 3 is laterally connected to the airway tube 1, detailed in FIG. 6 and FIG. 7. The carbon dioxide outlet 3 is in fluid communication with the airway tube 1 to allow carbon dioxide to be removed as the patient respires, in accordance to FIG. 8. The oxygen port 2 is preferred to be diametrically opposite the carbon dioxide outlet 3 about the airway tube 1 in order to prevent a hose connected to the oxygen port 2 from interfering with connecting another hose to the carbon dioxide outlet 3. The grasping flange 5 provides a hand hold for the user to manipulate the present invention onto the respiratory-device or to remove the present invention from the respiratory-device. The grasping flange 5 is perimetrically and terminally connected to the airway tube 1, detailed in FIG. 1 to FIG. 3 and FIG. 6 to FIG. 8. The implement port 4 is an opening that allows for the insertion, manipulation and removal of medical implements through the respiratory-device. The implement port 4 is centrally connected to the grasping flange 5, shown in FIG. 4. The implement port 4 is oppositely positioned to the airway tube 1 about the grasping flange 5, in accordance to FIG. 2, FIG. 3, and FIG. 6 to FIG. 8. The implement port 4 is in fluid communication with the airway tube 1, detailed in FIG. 8. The user is able to hold the grasping flange 5 in order to press the adapter plug 7 into the implement port 4, such that the adapter plug 7 restricts fluid flow through the implement port 4.

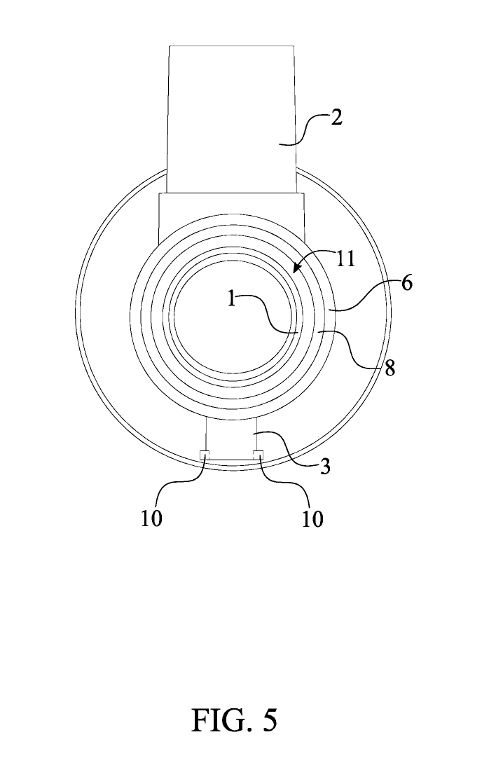

The mounting flange 6 and the adapter mounting tube 8 secure the present invention onto the respiratory-device. The mounting flange 6 is laterally and perimetrically connected to the airway tube 1, shown in FIG. 1. In accordance to FIG. 6 to FIG. 8, the oxygen port 2 and the carbon dioxide outlet 3 are positioned between the grasping flange 5 and the mounting flange 6 such that the oxygen port 2 and the carbon dioxide outlet 3, as well as the respective connected hoses, do not interfere with connecting the present invention with the respiratory-device or each other. The mounting flange 6 allows the adapter mounting tube 8 to be offset from the airway tube 1 to delineate a channel within which the respiratory-device is positioned in order to secure the present invention onto the respiratory-device. The adapter mounting tube 8 is adjacently connected to the mounting flange 6. The airway tube 1 is concentrically positioned within the adapter mounting tube 8. The adapter mounting tube 8 is oriented away from the oxygen port 2 and the carbon dioxide outlet 3. The airway tube 1, the mounting flange 6, and the adapter mounting tube 8 delineate a respiratory-device receiving channel 11, as the mounting flange 6 offsets the adapter mounting tube 8 from the airway tube 1, in accordance to FIG. 5 and FIG. 8. The respiratory-device is pushed into the respiratory-device receiving channel 11 in order to secure the present invention onto the respiratory-device. The adapter plug 7 seals the implement port 4 to prevent fluid flow through the implement port 4. The adapter plug 7 selectively engages the implement port 4.

Further in accordance to FIG. 1, the adapter plug 7 comprises a plug-mounting extrusion 12, a plug flange 13, an adapter orifice 14, and an implement-receiving channel 15. The plug-mounting extrusion 12 is centrally and normally connected to the plug flange 13. The plug-mounting extrusion 12 selectively engages the implement port 4 in order to seal the implement port 4 to prevent fluid flow to and from the airway tube 1 through the implement port 4. The adapter orifice 14 allows for implements with a smaller diameter than the diameter of the implement port 4 to be utilized by the user. The adapter orifice 14 traverses through the plug flange 13, shown in FIG. 3 and FIG. 8. The plug flange 13 presses against the implement that traverses through the adapter orifice 14 to seal the airway tube 1 from the atmosphere through the adapter orifice 14. The adapter orifice 14 is concentrically positioned with the plug-mounting extrusion 12. The implement-receiving channel 15 allows the adapter plug 7 to laterally engage an elongated implement such that the user is able to position the adapter plug 7 anywhere along the elongated implement. The implement-receiving channel 15 radially traverses through the plug flange 13, the plug-mounting extrusion 12, and into the adapter orifice 14, detailed in FIG. 1 and FIG. 3. Thus, the user is able to press the elongated implement through the implement-receiving channel 15, in order to position the elongated implement within the adapter orifice 14.

In accordance to the preferred embodiment, the adapter plug 7 further comprises an orifice plug 16, shown in FIG. 1. The orifice plug 16 allows the user to seal the adapter orifice 14 when an implement is not positioned through the adapter orifice 14. The orifice plug 16 is laterally mounted to the plug flange 13, such that the orifice plug 16 does not interfere with positioning the implement within the adapter orifice 14, detailed in FIG. 1, FIG. 6, and FIG. 7. The orifice plug 16 selectively engages the adapter orifice 14 to seal the adapter orifice 14 when the implement is not positioned within the adapter orifice 14.

Further in accordance to the preferred embodiment, the present invention comprises a plurality of tactile grooves 9, detailed in FIG. 1 and FIG. 8. The plurality of tactile grooves 9 improves the grip that the user has on the plug flange 13 when manipulating the adapter plug 7 onto or out from the implement port 4. The plurality of tactile grooves 9 traverses into the plug flange 13 in order to improve traction between the user's hand and the plug flange 13. The plurality of tactile grooves 9 is radially offset from the adapter orifice 14. Additionally, each tactile groove is radially offset from another. This configuration allows the plurality of tactile grooves 9 to effectively improve the user's grip radially across the plug flange 13.

In some embodiments of the present invention, the present invention further comprises a pair of tube mounting extrusions 10, shown in FIG. 3 and FIG. 5. The pair of tube mounting extrusions 10 assists in securing a carbon dioxide transfer hose onto the carbon dioxide outlet 3. The pair of tube mounting extrusions 10 is laterally connected to the carbon dioxide outlet 3. The pair of tube mounting extrusions 10 applies pressure to an interior wall of the carbon dioxide transfer hose as the carbon dioxide transfer tube is positioned around the carbon dioxide outlet 3. This pressure seals the carbon dioxide transfer tube against the pair of tube mounting extrusions 10 and the carbon dioxide outlet 3. The pair of tube mounting extrusions 10 is oppositely positioned to the airway tube 1 along the carbon dioxide outlet 3 in order to effectively engage the carbon dioxide transfer tube as the carbon dioxide transfer tube is positioned around the carbon dioxide outlet 3, in accordance to FIG. 6 and FIG. 7.

Although the invention has been explained in relation to its preferred embodiment, it is to be understood that many other possible modifications and variations can be made without departing from the spirit and scope of the invention as hereinafter claimed.

What is claimed is:

1. A removable fiberoptic adapter for difficult intubation with laryngeal mask airway comprises:
an airway tube;
an oxygen port;
a carbon dioxide outlet;
an implement inlet;
a grasping flange;
a mounting flange;
an adapter plug;
an adapter mounting tube;
the oxygen port being laterally connected to the airway tube;
the carbon dioxide outlet being laterally connected to the airway tube;
the grasping flange being perimetrically and terminally connected to the airway tube;
the implement inlet being centrally connected to the grasping flange;
the implement inlet being oppositely positioned to the airway tube about the grasping flange;
the mounting flange being laterally and perimetrically connected to the airway tube;
the oxygen port and the carbon dioxide outlet being positioned between the grasping flange and the mounting flange;
the adapter mounting tube being adjacently connected to the mounting flange;
the airway tube being concentrically positioned within the adapter mounting tube;
the adapter mounting tube being oriented away from the oxygen port and the carbon dioxide outlet; and
the adapter plug selectively engaging the implement inlet.

2. The removable fiberoptic adapter for difficult intubation with laryngeal mask airway, as claimed in claim 1, comprises:
the adapter plug comprises a plug-mounting extrusion, a plug flange, an adapter orifice, and an implement-receiving channel;
the plug-mounting extrusion being centrally and normally connected to the plug flange;
the adapter orifice traversing through the plug flange;
the adapter orifice being concentrically positioned within the plug-mounting extrusion;
the implement-receiving channel radially traversing through the plug flange, the plug-mounting extrusion, and into the adapter orifice; and
the plug-mounting extrusion selectively engaging the implement inlet.

3. The removable fiberoptic adapter for difficult intubation with laryngeal mask airway, as claimed in claim 2, comprises:
the adapter plug further comprises an orifice plug;
the orifice plug being laterally mounted to the plug flange; and
the orifice plug selectively engaging the adapter orifice.

4. The removable fiberoptic adapter for difficult intubation with laryngeal mask airway, as claimed in claim 2, comprises:
a plurality of tactile grooves;
the plurality of tactile grooves traversing into the plug flange;
the plurality of tactile grooves being radially offset from the adapter orifice; and
each tactile groove being radially offset from another.

5. The removable fiberoptic adapter for difficult intubation with laryngeal mask airway, as claimed in claim 1, comprises:
the oxygen port being diametrically opposed with the carbon dioxide outlet about the airway tube.

6. The removable fiberoptic adapter for difficult intubation with laryngeal mask airway, as claimed in claim 1, comprises:
the oxygen port being in fluid communication with the airway tube.

7. The removable fiberoptic adapter for difficult intubation with laryngeal mask airway, as claimed in claim 1, comprises:
the carbon dioxide outlet being in fluid communication with the airway tube.

8. The removable fiberoptic adapter for difficult intubation with laryngeal mask airway, as claimed in claim 1, comprises:
the implement inlet being in fluid communication with the airway tube.

9. The removable fiberoptic adapter for difficult intubation with laryngeal mask airway, as claimed in claim 1, comprises:
a pair of tube mounting extrusions;
the pair of tube mounting extrusions being laterally connected to the carbon dioxide outlet; and
the pair of tube mounting extrusions being oppositely positioned to the airway tube along the carbon dioxide outlet.

10. The removable fiberoptic adapter for difficult intubation with laryngeal mask airway, as claimed in claim 1, comprises:
the airway tube, the mounting flange, and the adapter mounting tube delineating a respiratory-device receiving channel.

11. A removable fiberoptic adapter for difficult intubation with laryngeal mask airway comprises:
an airway tube;
an oxygen port;
a carbon dioxide outlet;
an implement inlet;
a grasping flange;
a mounting flange;
an adapter plug;
the adapter plug comprises a plug-mounting extrusion, a plug flange, an adapter orifice, and an implement-receiving channel;
an adapter mounting tube;
the oxygen port being laterally connected to the airway tube;
the carbon dioxide outlet being laterally connected to the airway tube;
the grasping flange being perimetrically and terminally connected to the airway tube;
the implement inlet being centrally connected to the grasping flange;
the implement inlet being oppositely positioned to the airway tube about the grasping flange;
the mounting flange being laterally and perimetrically connected to the airway tube;
the oxygen port and the carbon dioxide outlet being positioned between the grasping flange and the mounting flange;
the adapter mounting tube being adjacently connected to the mounting flange;
the airway tube being concentrically positioned within the adapter mounting tube;
the adapter mounting tube being oriented away from the oxygen port and the carbon dioxide outlet;
the adapter plug selectively engaging the implement inlet;
the plug-mounting extrusion being centrally and normally connected to the plug flange;
the adapter orifice traversing through the plug flange;
the adapter orifice being concentrically positioned within the plug-mounting extrusion;
the implement-receiving channel radially traversing through the plug flange, the plug-mounting extrusion, and into the adapter orifice; and
the plug-mounting extrusion selectively engaging the implement inlet.

12. The removable fiberoptic adapter for difficult intubation with laryngeal mask airway, as claimed in claim 11, comprises:
the adapter plug further comprises an orifice plug;
the orifice plug being laterally mounted to the plug flange; and
the orifice plug selectively engaging the adapter orifice.

13. The removable fiberoptic adapter for difficult intubation with laryngeal mask airway, as claimed in claim 11, comprises:
a plurality of tactile grooves;
the plurality of tactile grooves traversing into the plug flange;
the plurality of tactile grooves being radially offset from the adapter orifice; and
each tactile groove being radially offset from another.

14. The removable fiberoptic adapter for difficult intubation with laryngeal mask airway, as claimed in claim 11, comprises:
the oxygen port being diametrically opposed with the carbon dioxide outlet about the airway tube.

15. The removable fiberoptic adapter for difficult intubation with laryngeal mask airway, as claimed in claim 11, comprises:
the oxygen port being in fluid communication with the airway tube.

16. The removable fiberoptic adapter for difficult intubation with laryngeal mask airway, as claimed in claim 11, comprises:
the carbon dioxide outlet being in fluid communication with the airway tube.

17. The removable fiberoptic adapter for difficult intubation with laryngeal mask airway, as claimed in claim 11, comprises:
the implement inlet being in fluid communication with the airway tube.

18. The removable fiberoptic adapter for difficult intubation with laryngeal mask airway, as claimed in claim 11, comprises:
a pair of tube mounting extrusions;
the pair of tube mounting extrusions being laterally connected to the carbon dioxide outlet; and
the pair of tube mounting extrusions being oppositely positioned to the airway tube along the carbon dioxide outlet.

19. The removable fiberoptic adapter for difficult intubation with laryngeal mask airway, as claimed in claim 11, comprises:
the airway tube, the mounting flange, and the adapter mounting tube delineating a respiratory-device receiving channel.
